(19) United States
(12) Patent Application Publication
(10) Pub. No.: US 2026/0191841 A1
(43) Pub. Date: Jul. 9, 2026

(54) TREATMENT OF CHRONIC KIDNEY DISEASE IN TYPE I DIABETES MELLITUS

(71) Applicant: BAYER AKTIENGESELLSCHAFT, Leverkusen (DE)

(72) Inventors: Markus Florian SCHEERER, München (DE); Peter KOLKHOF, Wuppertal (DE); Robert EDFORS, Bromma (SE); Robert LAWATSCHECK, Berlin (DE); Meike Daniela BRINKER, Düsseldorf (DE)

(21) Appl. No.: 19/132,218

(22) PCT Filed: Nov. 22, 2023
(86) PCT No.: PCT/EP2023/082668

§ 371 Date: May 22, 2025

Related U.S. Application Data
(60) Provisional application No. 63/384,910, filed on Nov. 23, 2022.
(60) Provisional application No. 63/515,029, filed on Jul. 21, 2023.

(51) Int. Cl. A61K 31/4375; A61K 45/06; A61P 13/12

(52) U.S. Cl. CPC A61K 31/4375; A61K 45/06; A61P 13/12

(57) ABSTRACT

The invention also refers to finerenone or a hydrate, solvate, pharmaceutically acceptable salt thereof, or a polymorph thereof for use in the prevention or treatment of chronic kidney disease in a patient with type I diabetes, comprising administering to the patient a therapeutically effective amount of finerenone or a hydrate, solvate, pharmaceutically acceptable salt thereof, or a polymorph thereof.

The present invention concerns medical therapy for preventing, ameliorating, or treating chronic kidney disease in type I diabetes mellitus. The invention also refers to finerenone or a hydrate, solvate, pharmaceutically acceptable salt thereof, or a polymorph thereof for use in the prevention or treatment of chronic kidney disease in a patient with type I diabetes, comprising administering to the patient a therapeutically effective amount of finerenone or a hydrate, solvate, pharmaceutically acceptable salt thereof, or a polymorph thereof.

Diabetes mellitus is a chronic disorder characterized by hyperglycemia resulting from defects in insulin secretion, insulin action, or both. Type 1 diabetes mellitus (TID) is characterized by little or no insulin secretory capacity, and patients with type 1 diabetes mellitus require insulin for survival. Because Type 1 diabetes patients produce little or no insulin, effective insulin therapy for Type 1 diabetics generally involves the use of two types of exogenously administered insulin: a rapid-acting, mealtime insulin provided by bolus injections, insulin pumps, closed loop systems, inhaled insulins, and a long-acting, basal insulin, administered once or twice daily to control blood glucose levels between meals.

In the United States approximately 1.3 million people are diagnosed with type 1 diabetes. T1D is the one of the most common diseases of childhood and requires lifelong insulin administration. Among the US population, a 2019 study cited by the Diabetes in America Report by NIH/NIDDK estimates a prevalence of 740,000 to 970,000 individuals with T1D in the US between 1999 and 2010 and it is anticipated that it may triple by 2050 due to a rising incidence of TID. (Imperatore G. et al. Diabetes Care. 2012, 35(12):2515-2520). An estimate in 2016 by the United States Centers for Disease Control and Prevention (CDC) indicated that 1,300,000 adults (≥18 years)—or 5.8% of adults diagnosed with diabetes and 0.55% of all US adults—reported both having T1D and using insulin. (Bullard K M, et al. Morb Mortal Wkly Rep. 2018, 67(12):359-361). CDC data from 2019 showed that 244,000 children and adolescents (<20 years) in the US had diagnosed type 1 diabetes. (Menke A, et al. Epidemiology. 2013, 24(5):773-774).

Chronic kidney disease (CKD) is a main complication of TID. CKD is often asymptomatic until later stages, when non-specific symptoms such as lethargy, itch, or loss of appetite arise. Diagnosis is commonly made after chance findings from screening tests (e.g., laboratory measurement, urinary dipstick or blood tests), or in late-stage CKD when symptoms begin to manifest.

The down-stream effects of hyperglycemia are the pathophysiologic drivers of CKD in TID. Chronically elevated blood glucose levels can lead to albuminuria or elevated urine albumin-to-creatinine ratio (UACR) levels, hypertension, micro- and macrovascular lesions, oxidative stress and inflammation and fibrosis in the kidneys as well as elevated risk for cardiovascular (CV) events. (Papadopoulou-Marketou N, et al. Diabetes Metab Res Rev. 2017, 33(2); Nathan D M et al. Diabetes Care. 2014, 37(1):9-16). Higher albuminuria and lower eGFR are independent risk factors for progression of CKD and end-stage kidney disease (ESKD) or end-stage renal diseases (ESRD) as well as CV morbidity and mortality (Gansevoort R T et al. Kidney Int. 2011, 80(1):93-104; Matsushita K et al. Lancet. 2010, 375(9731):2073-2081; van der Velde M et al. Kidney Int. 2011; 79(12):1341-1352). The term end-stage kidney disease (ESKD) can be used synonymously with end-stage renal diseases (ESRD). The term “kidney” can be used synonymously with “renal”.

In a systemic analysis for the Global Burden of Disease study, it was reported that the prevalence of CKD due to T1D had increased by 58.2% from 1990 to 2007 and by 21.7% from 2007 to 2017. The 2017 global prevalence of CKD due to T1D was an estimated 32.5 per 100,000 individuals. (James, S L et al. Lancet 2018, 392(10159):1789-1858). The frequency of CKD in T1D in adults in the US is approximately 25-40% and 25-30% of patients with T1D develop end-stage kidney or renal disease. (Bakris G L, Diabetes Care. 2018, 41(3):389-390).

The current treatment of T1D consists of insulin treatment to control hyperglycemia. In patients with TID, blood glucose intervention targeting HbA1c levels≤7% can slow onset and progression of kidney disease. Treatment with ACE-inhibitors (ACEI) or Angiotensin Receptor Blockers (ARB) is often required to control blood pressure and to reduce albuminuria to slow the progression of established kidney disease. In agents targeting the renin-angiotensin-aldosterone system, like ACEI and ARB, a reduction of albuminuria was shown to be in line with a reduction of clinical outcomes, such as progression to ESKD or ESRD for both types of diabetes. (Brenner B M et al. N Engl J Med. 2001, 345(12):861-869; Lewis, E J, et al. N Engl J Med. 1993, 329(20):1456-1462; Björck S et al. BMJ. 1992, 304(6823):339-343; Lewis E J et al. N Engl J Med. 2001; 345(12):851-860).

Although sodium-glucose cotransporter-2 (SGLT2) inhibitors are prescribed as adjuvants to insulin to improve blood glucose control and reduce total daily insulin dose and body weight in TID, this class of drugs is of limited use in clinical practice due to safety concern of diabetic ketoacidosis. Furthermore, SGLT2 inhibitors are not currently approved for the treatment of T1D and CKD by European Medicines Agency (EMA) or the US Food and Drug Administration (FDA). Despite these current guideline recommended therapies with insulin and ACEI or ARB, a quarter of patients with CKD and T1D progress to ESKD or ESRD and dialysis and CKD is still the leading cause for cardio-vascular events as well as mortality in T1D.

No new treatments for CKD and T1D have emerged since the ACEI and ARB studies almost 30 years ago. For example, the landmark captopril trial was long ago, in 1993. (Lewis E J et al. NEJM 1993; 329:1456-1462). Additionally, once promising reno-protective strategies for CKD and T1D such as protein kinase C-β antagonists, selective endothelin receptor-A antagonists, and the Nrf2 modulator bardoxolone have resulted in disappointing clinical trial results. (Cherney D Z I et al. Diabetes Care. 2010, 33(6):1344-1346). Recently, in the Preventing Early Renal Loss in Diabetes Study, which studied the effect of lowering uric acid in patients with CKD and TID, allopurinol did not reduce albuminuria nor delay the rate of kidney function decline vs placebo. (Doria A, et al. N Engl J Med. 2020, 382(26):2493-2503).

An object of the present invention is to provide a new intervention to delay or prevent the progression of CKD in T1D.

A further object of the invention is to provide a new intervention to extend the healthy life span, and reduce the mortality and/or morbidity in patients with CKD and T1D.

The non-steroidal mineralocorticoid receptor antagonist (MRA) finerenone improves renal and cardiovascular function in patients with CKD in type 2 diabetes (T2D). Finerenone lessens the effects of mineralocorticoid receptor overactivation by ligands such as aldosterone and cortisol. Finerenone has the chemical name (4S)-4-(4-cyano-2-methoxyphenyl)-5-ethoxy-2,8-dimethyl-1,4-dihydro-1,6-naphthyridine-3-carboxamide, and has the chemical structure of formula (I):

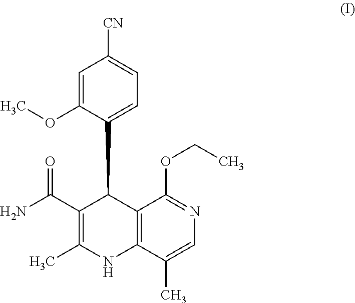 [SMILES: CCOc1ncc(C)c2c1[C@H](c1ccc(C#N)cc1OC)C(C(N)=O)=C(C)N2]

The synthesis, pharmacological properties, and pharmaceutical formulations/dosage forms of finerenone are described in U.S. Pat. No. 8,436,180, which is hereby incorporated by reference herein in its entirety.

The effects of finerenone on various aspects of diabetic kidney disease in T2D have been studied. For example, it has been found that finerenone coadministered with an ACEI or an ARB delays the progression of diabetic kidney disease and reduces cardiovascular morbidity in patients with T2D. (Veneti S. et al. Diabetes Therapy. 2021, 12(7), 1791-1797).

It has been proposed that a combination of specific pharmacokinetic and pharmacodynamic properties including tissue distribution, selectivity, and binding mode of finerenone to the mineralocorticoid receptor (MR) is the basis for an improved benefit/risk profile including a reduced risk for developing hyperkalemia and renal dysfunction in patients with heart failure and diabetic kidney disease. Studies conducted in patients with heart failure with reduced ejection fraction (HFrEF) and moderate chronic kidney disease and diabetic kidney disease showed promising results. (Licette, C Y et al. Expert Opinion on Investigational Drugs. 2015, 24(8), 1123-1135).

The effects of finerenone have been explored in a novel model of CKD in T1D using a Munich Wistar Frömter (MWF) rat model. (Sanz-Gómez, M et al. Journal of Hypertension. 2022, Vol. 40, Issue Suppl. 1, p. e37).

To determine whether mineralocorticoid receptor (MR) activation plays a role in diabetic renal injury in types 1 and 2 diabetes mellitus, the effect was measured of the MR antagonist eplerenone on renal injury in rodent models of type 1 (streptozotocin-treated rat) and type 2 (db/db mouse) diabetes. (Guo et al., Endocrinology 2006, 147(11):5363-5373). From these eplerenone studies, Guo et al. suggest that MR activation is a critical factor in the early pathogenesis of renal disease in both type 1 and type 2 diabetes mellitus.

In an embodiment, the disclosure concerns a method of preventing or treating chronic kidney disease in a patient with type I diabetes, comprising administering to the patient a therapeutically effective amount of finerenone or a hydrate, solvate, pharmaceutically acceptable salt thereof, or a polymorph thereof. In an embodiment, the disclosure concerns a method to delay the progression of chronic kidney disease in a patient with chronic kidney disease associated with type 1 diabetes comprising administering to the patient a therapeutically effective amount of finerenone or a hydrate, solvate, pharmaceutically acceptable salt thereof, or a polymorph thereof. Additionally, the disclosure concerns a method to reduce the risk of sustained UACR decline in a patient with chronic kidney disease associated with type 1 diabetes comprising administering to the patient a therapeutically effective amount of finerenone or a hydrate, solvate, pharmaceutically acceptable salt thereof, or a polymorph thereof. In another embodiment, the disclosure concerns a method to reduce UACR in a patient with chronic kidney disease associated with type 1 diabetes comprising administering to the patient a therapeutically effective amount of finerenone or a hydrate, solvate, pharmaceutically acceptable salt thereof, or a polymorph thereof. In any of the embodiments of the present disclosure, the disclosure further concerns reducing the risk of end stage kidney disease or renal diseases.

The invention refers to methods of treating, preventing, and delaying progression of CKD in T1D or associated conditions thereof by administering finerenone or a hydrate, solvate, pharmaceutically acceptable salt thereof, or a polymorph thereof. The methods disclosed herein for use with finerenone also may be used with therapeutically effective amounts of a hydrate, solvate, pharmaceutically acceptable salt or polymorph of finerenone.

Polymorphic form of finerenone is disclosed in U.S. Pat. No. 10,399,977, which is hereby incorporated herein by reference in its entirety. In the methods of the present disclosure, the compound to be administered may be finerenone of the formula (I) in crystalline form of polymorph I characterized in that the x-ray diffractogram of the compound exhibits peak maxima of the 2 theta angle at 8.5, 14.1, 17.2, 19.0, 20.5, 25.6, 26.5. In the methods of the present disclosure, the compound to be administered may be finerenone of the formula (I) in crystalline form of polymorph I characterized in that the IR spectrum (IR-ATR) of the compound exhibits band maxima at 3475, 2230, 1681, 1658, 1606, 1572, 1485, 1255, 1136 and 1031 cm⁻¹. In the methods of the present disclosure, the compound to be administered may be finerenone of the formula (I) in crystalline form of polymorph I characterized in that the Raman spectrum of the compound exhibits band maxima at 3074, 2920, 2231, 1601, 1577, 1443, 1327, 1267, 827 and 155 cm⁻¹. Experimental conditions for the measurement of these crystalline form parameters are found in the examples.

In one embodiment, finerenone of the formula (I) in crystalline form of polymorph I is used. In one embodiment, finerenone is the compound of the formula (I) in crystalline form of polymorph I

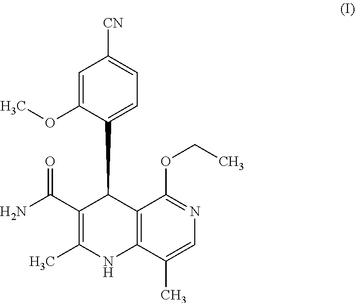 [SMILES: CCOc1ncc(C)c2c1[C@H](c1ccc(C#N)cc1OC)C(C(N)=O)=C(C)N2]

wherein the x-ray diffractogram of the compound exhibits peak maxima of the 2 theta angle at 8.5, 14.1, and 19.0. In one embodiment, finerenone is the compound of the formula (I) in crystalline form of polymorph I, wherein the x-ray diffractogram of the compound further exhibits any one f peak maxima of the 2 theta angle at 17.2, 20.5, 25.6, and 26.5. In one embodiment, finerenone is the compound of the formula (I) in crystalline form of polymorph I, wherein the IR spectrum of the compound exhibits any one of band maxima at 3475, 2230, 1681, 1658, 1606, 1572, 1485, 1255, 1136 and 1031 cm⁻¹. In one embodiment, finerenone is the compound of the formula (I) in crystalline form of polymorph I, wherein the IR spectrum of the compound exhibits band maxima at 3475, 2230, 1681, 1658, 1606, 1572, 1485, 1255, 1136 and 1031 cm⁻¹. In one embodiment, finerenone is the compound of the formula (I) in crystalline form of polymorph I, wherein the Raman spectrum of the compound exhibits any one of band maxima at 3074, 2920, 2231, 1601, 1577, 1443, 1327, 1267, 827 and 155 cm⁻¹. In one embodiment, finerenone is the compound of the formula (I) in crystalline form of polymorph I, wherein the Raman spectrum of the compound exhibits band maxima at 3074, 2920, 2231, 1601, 1577, 1443, 1327, 1267, 827 and 155 cm⁻¹. In one embodiment, finerenone is the compound of the formula (I) in crystalline form of polymorph I, wherein the compound has a melting point of 252° C. In one embodiment, the invention concerns a safe and effective method of preventing or treating chronic kidney disease in a patient with type I diabetes, including administering to the patient a therapeutically effective amount of finerenone. In another embodiment, the invention concerns a safe and effective method to delay the progression of chronic kidney disease in a patient with chronic kidney disease associated with type 1 diabetes including administering to the patient a therapeutically effective amount of finerenone. In certain embodiments, the method attenuates kidney function decline and progression to end stage kidney disease or end stage renal diseases in a patient with chronic kidney disease associated with type 1 diabetes by administering to the patient a therapeutically effective amount of finerenone. In further embodiments, the invention concerns a safe and effective method to reduce the risk of sustained UACR decline in a patient with chronic kidney disease associated with type 1 diabetes by administering to the patient a therapeutically effective amount of finerenone. In further embodiments, the invention concerns a safe and effective method to reduce the risk of sustained UACR decline in a patient with CKD associated with type 1 diabetes by administering to the patient a therapeutically effective amount of finerenone. In further embodiments, the invention concerns a safe and effective method to reduce albuminuria in a patient with end stage kidney disease or end stage renal diseases associated with type 1 diabetes comprising administering to the patient a therapeutically effective amount of finerenone.

In further embodiments, the invention concerns a safe and effective method to provide a statistically significant reduction of UACR (=>0.05 p-value) in a patient with CKD and T1D comprising administering to the patient a therapeutically effective amount of finerenone. Statistical analyses may be performed with the use of SAS software, version 9.4 or later (SAS Institute). In one embodiment, the placebo-corrected relative reduction in UACR in patients receiving finerenone (dosage of 10 mg or 20 mg) is statistically significant at a 95% CI after 6 months of administration of finerenone.

The daily dosage of finerenone can be selected from the group consisting of 7.5 mg, 10 mg, 20 mg, 40 mg, a range of 10 mg to 20 mg, and a range of 10 mg to 40 mg. One embodiment according to the invention refers to administering finerenone in a daily amount of 0.25 mg to 80 mg. One embodiment according to the invention refers to administering finerenone in a daily amount of 0.25 mg to 40 mg. Other embodiments according to the invention refer to administering finerenone in a daily amount of 0.25 mg to 20 mg, 0.25 mg to 10 mg, or 0.25 mg to 5 mg. In one embodiment the dosage of finerenone is titrated during the study with a target dose of 10 mg. In one embodiment the dosage of finerenone is titrated during the study with a target dose of 20 mg. In one embodiment the dosage of finerenone is titrated during the study with a target dose of 40 mg.

Other embodiments according to the invention refer to administering finerenone in an amount of 5 to 80 mg, 5 to 70 mg, 5 to 60 mg, 5 to 50 mg, 5 to 40 mg, 10 to 80 mg, 10 to 70 mg, 10 to 60 mg, 10 to 50 mg or 10 to 40 mg. Other embodiments according to the invention refer to administering a daily dosage of finerenone in an amount of 10 to 40 mg, or 20 to 40 mg. In one embodiment the methods according to the invention comprise administering finerenone in an amount of 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 7.5, 10, 15, 20, 25, 30, 35, 40, 45, 50, 55, 60, 65, 70, 75 or 80 mg. In one embodiment the inventive method comprises administering finerenone in an amount of 5 mg. In one embodiment the inventive method comprises administering finerenone in an amount of 10 mg. In one embodiment the inventive method comprises administering finerenone in an amount of 15 mg. In one embodiment the inventive method comprises administering finerenone in an amount of 20 mg. In one embodiment the inventive method comprises administering finerenone in an amount of 25 mg. In one embodiment the inventive method comprises administering finerenone in an amount of 30 mg. In one embodiment the inventive method comprises administering finerenone in an amount of 35 mg. In one embodiment the inventive method comprises administering finerenone in an amount of 40 mg. In one embodiment the inventive method comprises administering finerenone in an amount of 5 mg, 7.5 mg, 10 mg, 15 mg, 20 mg, 25 mg, 30 mg, 35 mg or 40 mg. In one embodiment the inventive method comprises administering finerenone in an amount of 5 mg, 10 mg, 20 mg, 30 mg or 40 mg.

Formulations of finerenone are known in the art and include the formulations disclosed in U.S. Pat. No. 8,436,180. Common dosage forms include pill, tablet, capsule, syrup, aerosol, liquid injection, powder, or solid crystal. Finerenone is preferably administered orally in a solid dosage form. Formulations of finerenone may include inactive ingredients such as lactose monohydrate, cellulose microcrystalline, croscarmellose sodium, hypromellose, magnesium stearate, and sodium lauryl sulfate. The film coating on a finerenone tablet may contain hypromellose, titanium dioxide and talc, in addition to ferric oxide red (10 mg strength tablets) or ferric oxide yellow (20 mg strength tablets).

Finerenone is commercially available. In one embodiment, finerenone is present in film-coated tablets administered orally at a dosage of 10 mg once daily or 20 mg once daily. In some embodiments, a patient initiates treatment with an oral dosage of 10 mg once daily and then is up-titrated to a maintenance dosage of 20 mg orally once daily. In some embodiments, a patient initiates treatment with an oral dosage of 10 mg once daily and then is up-titrated to a maintenance dosage of 20 mg orally once daily after one month or 3-5 weeks of receiving the lower initial dosage.

In one embodiment, finerenone is present in film-coated tablets administered orally at a dosage of 40 mg once daily. In some embodiments, a patient initiates treatment with an oral dosage of 10 mg once daily and then is up-titrated to a maintenance dosage of 20 mg orally once daily or 40 mg orally daily. In some embodiments, a patient initiates treatment with an oral dosage of 10 mg once daily and then is up-titrated to a maintenance dosage of 20 mg orally once daily or 40 mg orally daily, after one month or 3-5 weeks of receiving the lower initial dosage.

Preferably the tablets are immediate-release formulations. In one embodiment, finerenone is administered in the morning at approximately the same time each day for the duration of effective treatment. Finerenone may be taken with or without food.

In an embodiment, the patient with T1D may also be receiving insulin in addition to finerenone. In an aspect, the proposed method further includes that for >4 weeks prior to initiating finerenone therapy, the patient has been administered a stable dosage of ACEI or ARB. ACEIs are known in the art and include without limitation captopril, enalapril, lisinopril, benazepril, fosinopril, quinapril, ramipril, perindopril, moexipril and trandolapril. Angiotensin II receptor blockers (ARBs) are known in the art and include without limitation losartan, valsartan, telmisartan, irbesartan, azilsartan, and olmesartan. The patient may be receiving insulin as well as the stable dosage of ACEI or ARB. In certain embodiments, one or more of the following treatments do not occur in a patient receiving the therapy of the present disclosure: concomitant therapy with both an ACEI and an ARB; concomitant treatment with a SGLT-2/-1 inhibitor or GLP1 receptor agonist, concomitant treatment with strong CYP3A4 inhibitors, concomitant treatment with moderate/strong CYP3A4 inducers, concomitant therapy with an MRA other than finerenone, and/or concomitant therapy with any renin inhibitor, sacubitril/valsartan combination, or potassium-sparing diuretic. Examples of moderate CYP3A4 inhibitors include amiodarone, aprepitant, bicalutamide, chloramphenicol, imatinib, mifepristone, norfloxacine, tacrolimus, verapamil, lapatinib, dasatinib and nilotinib.

According to an aspect of the invention, the proposed method further includes evaluating the UACR of the patient prior to receiving finerenone. A baseline UACR for the patient can be measured before beginning the administration of finerenone. The baseline UACR can be measured at the time of first administration of finerenone to the patient. According to the inventive methods, the patient can have (1) albuminuria, (2) a UACR of equal to or above 150, 200, 250, 300, 350, 400, 450 or 500 mg/g, and/or (3) a UACR of 30 to 5000, 200 to 5000, 200 to 4000, 200 to 3000, or 200 to 2000 mg/g. In one embodiment, the patient has a UACR of 200 to 5000 mg/g prior to administration of finerenone. In one embodiment, the patient has a UACR of 30 to 1000, 200 to 1000, 200 to 1000 mg/g prior to administration of finerenone. In one embodiment, the patient has a UACR of 300 to 1000, 300 to 5000, 300 to 4000, 300 to 3000, or 300 to 2000 mg/g.

In one embodiment, the patient has a UACR of equal to or below 200 mg/g prior to administration of finerenone. In one embodiment, the patient has a UACR of equal to or above 200 mg/g prior to administration of finerenone. In one embodiment, the patient has a UACR of equal to or below 300 mg/g prior to administration of finerenone. In one embodiment, the patient has a UACR of equal to or above 300 mg/g prior to administration of finerenone. In one embodiment, the patient has a UACR of 200 to 5000 mg/g prior to administration of finerenone. In one embodiment, the patient has a UACR of 300 to 1000 mg/g prior to administration of finerenone. In one embodiment, the patient has a UACR of 200 to 1000 mg/g prior to administration of finerenone. In one embodiment, the patient has a UACR of 400 to 5000 mg/g prior to administration of finerenone. In one embodiment, the patient has a UACR of equal to or below 1000 mg/g prior to administration of finerenone. In one embodiment, the patient has a UACR of equal to or above 1000 mg/g prior to administration of finerenone. In one embodiment, the patient has a UACR of equal to or below 5000 mg/g prior to administration of finerenone. In one embodiment, the patient has a UACR of equal to or above 5000 mg/g prior to administration of finerenone.

In one embodiment, the patient has (1) albuminuria, and (2) a UACR of equal to or below 200 mg/g prior to administration of finerenone. In one embodiment, the patient has (1) albuminuria, and (2) a UACR of equal to or above 200 mg/g prior to administration of finerenone. In one embodiment, the patient has (1) albuminuria, and (2) a UACR of equal to or below 300 mg/g prior to administration of finerenone. In one embodiment, the patient has (1) albuminuria, and (2) a UACR of equal to or above 300 mg/g prior to administration of finerenone. In one embodiment, the patient has (1) albuminuria, and (2) a UACR of 200 to 5000 mg/g prior to administration of finerenone. In one embodiment, the patient has (1) albuminuria, and (2) a UACR of 300 to 1000 mg/g prior to administration of finerenone. In one embodiment, the patient has (1) albuminuria, and (2) a UACR of 200 to 1000 mg/g prior to administration of finerenone. In one embodiment, the patient has (1) albuminuria, and (2) a UACR of 400 to 5000 mg/g prior to administration of finerenone. In one embodiment, the patient has (1) albuminuria, and (2) a UACR of equal to or below 1000 mg/g prior to administration of finerenone. In one embodiment, the patient has (1) albuminuria, and (2) a UACR of equal to or above 1000 mg/g prior to administration of finerenone. In one embodiment, the patient has (1) albuminuria, and (2) a UACR of equal to or below 5000 mg/g prior to administration of finerenone. In one embodiment, the patient has (1) albuminuria, and (2) a UACR of equal to or above 5000 mg/g prior to administration of finerenone.

In one embodiment, the patient has
(1) albuminuria, and
(2) a UACR of equal to or below 300 mg/g, a UACR of equal to or above 300 mg/g, a UACR of 300 to 1000 mg/g, a UACR of equal to or below 1000 mg/g or a UACR of equal to or above 1000 mg/g.

In one embodiment, the patient has (1) albuminuria, and (2) a UACR of equal or below 300 mg/g prior to administration of finerenone. In one embodiment, the patient has (1) albuminuria, and (2) a UACR of equal or above 300 mg/g prior to administration of finerenone. In one embodiment, the patient has (1) albuminuria, and (2) a UACR of 300 to 1000 mg/g prior to administration of finerenone. In one embodiment, the patient has (1) albuminuria, and a UACR of equal to or below 1000 mg/g prior to administration of finerenone. In one embodiment, the patient has (1) albuminuria, and a UACR of equal to or above 1000 mg/g prior to administration of finerenone.

As used herein, the UACR values are mean values. In other embodiments the disclosed methods are administered to patients with CKD and T1D without any preselection of patients by UACR levels or presence of albuminuria.

According to another aspect of the invention, the proposed method further includes evaluating the eGFR of the patient prior to receiving finerenone. A baseline eGFR level for the patient can be measured before beginning the administration of the finerenone. The baseline eGFR can be measured at the time of first administration of finerenone to the patient.

The baseline eGFR can be measured at the time of first administration of finerenone to the patient. The eGFR level can be determined using a blood test for a creatine level. The patient can have (1) an eGFR of equal to or above 5, 10, 15, 20, 25, 30, 35, 40, 45, 50, 55, 60, 65, 70, or 75 mL/min/1.73 m², or (2) an eGFR of from 15-90, 20-90, 25-90, 60-90, 25-75, or 25-60 mL/min/1.73 m².

In one embodiment, the patient has an eGFR of 25 to <90 ml/min/1.73 m². In another embodiment, prior to receiving finerenone or concomitant with initiation of finerenone therapy, the patient with type I diabetes has a UACR of 200 to 5000 mg/g, an eGFR of 25 to <90 ml/min/1.73 m², and has been administered a stable dosage of an ACEI or ARB for >4 weeks.

The eGFR level can be determined using a blood test for a creatine level. In some embodiments, the patient has an eGFR equal to or above 5, 10, 15, 20, 25, 30, 35, 40, 45, 50, 55, 60, 65, 70, or 75 mL/min/1.73 m² that preferably also is below 90 mL/min/1.73 m². In further embodiments, the patient has an eGFR of from 15-90, 20-90, 25-90, 30-90, 60-90, 25-75, or 25-60 mL/min/1.73 m². In one embodiment, the patient has an eGFR equal to or below 5, 10, 15, 20, 25, 30, 35, 40, 45, 50, 55, 60, 65, 70, or 75 mL/min/1.73 m². In one embodiment, the patient has an eGFR of 25 to <90 ml/min/1.73 m².

In one embodiment, prior to receiving finerenone, the patient has an eGFR of 15 to 60 ml/min/1.73 m². In one embodiment, prior to receiving finerenone, the patient has an eGFR of 20 to 60 ml/min/1.73 m². In one embodiment, prior to receiving finerenone, the patient has an eGFR of 25 to 60 ml/min/1.73 m². In one embodiment, prior to receiving finerenone, the patient has an eGFR of 30 to 60 ml/min/1.73 m². In one embodiment, prior to receiving finerenone, the patient has an eGFR of 45 to 60 ml/min/1.73 m². In one embodiment, the patient has an eGFR of 60 to 75 ml/min/1.73 m². In one embodiment, prior to receiving finerenone, the patient has an eGFR of 60 to 90 ml/min/1.73 m². In one embodiment, prior to receiving finerenone, the patient has an eGFR of 15 to 90 ml/min/1.73 m². In one embodiment, prior to receiving finerenone, the patient has an eGFR of 20 to 90 ml/min/1.73 m². In one embodiment, prior to receiving finerenone, the patient has an eGFR of 25 to 90 ml/min/1.73 m². In one embodiment, prior to receiving finerenone, the patient has an eGFR of 30 to 90 ml/min/1.73 m². In one embodiment, prior to receiving finerenone, the patient has an eGFR of 45 to 90 ml/min/1.73 m².

In one embodiment, the patient has an eGFR of equal to or below 45 mL/min/1.73 m². In one embodiment, the patient has an eGFR of equal to or above 45 mL/min/1.73 m². In one embodiment, the patient has an eGFR of 45 to 60 ml/min/1.73 m². In one embodiment, the patient has an eGFR of equal or below 60 ml/min/1.73 m². In one embodiment, the patient has an eGFR of equal or above 60 ml/min/1.73 m².

In another embodiment, prior to receiving finerenone or concomitant with initiation of finerenone therapy, the patient with T1D has a UACR of 200 to 5000 mg/g, an eGFR of 25 to <90 ml/min/1.73 m², and has been administered a stable dosage of an ACEI or ARB for at least 4 weeks, at least 3 weeks, at least 2 weeks, or about 2-4 weeks before initiation of finerenone therapy.

In one embodiment, the patient has an eGFR of <90 ml/min/1.73 m².

In another embodiment, prior to receiving finerenone or concomitant with initiation of finerenone therapy, the patient with T1D has a UACR of 200 to 5000 mg/g, an eGFR of 25 to <90 ml/min/1.73 m², and has been administered a stable dosage of an ACEI or ARB for at least 4 weeks.

One embodiment is a method of preventing or treating CKD in a patient with T1D comprising administering to the patient a therapeutically effective amount of finerenone or a hydrate, solvate, pharmaceutically acceptable salt thereof, or a polymorph thereof, wherein prior to receiving finerenone, the patient has
(1) an eGFR equal to or below 5, 10, 15, 20, 25, 30, 35, 40, 45, 50, 55, 60, 65, 70, or 75 ml/min/1.73 m², or
(2) an eGFR of from 15-90, 20-90, 25-90, 30-90, 60-90, 25-75, or 25-60 ml/min/1.73 m².

In one embodiment the patient prior receiving finerenone, the patient has an
(1) a UACR of
   equal to or below 200 mg/g, or
   equal to or above 200 mg/g, or
   equal to or below 300 mg/g, or
   equal to or above 300 mg/g, or
   300 to 1000 mg/g, or
   equal to or below 1000 mg/g, or
   equal to or above 1000 mg/g, or
   200 to 5000 mg/g, or
   200 to 1000 mg/g, or
   equal to or below 5000 mg/g.
and
(2) an eGFR of
   equal to or below 25 mL/min/1.73 m², or
   equal to or above 25 mL/min/1.73 m², or
   equal to or below 45 mL/min/1.73 m², or
   equal to or above 45 mL/min/1.73 m², or
   45 to 60 mL/min/1.73 m², or
   45 to 90 mL/min/1.73 m², or
   25 to 60 mL/min/1.73 m², or
   25 to 90 mL/min/1.73 m², or
   equal or below 60 mL/min/1.73 m², or
   equal or above 60 mL/min/1.73 m², or
   equal or below 90 mL/min/1.73 m².

In further embodiments, the finerenone can be administered to the patient for at least three months, or at least six months. In an embodiment, after administration of finerenone for at least three months or at least 6 months, the patient has substantial reduction in UACR over baseline. In other embodiments the patient has a reduction of at least 20%, 25%, 30%, 35%, 40%, or 50% of UACR over baseline after administration of finerenone, after administration of finerenone for at least 3 months, or after administration of finerenone for at least 6 months.

The methods of the invention can be used for T1D patients having or at risk of developing CKD, particularly patients having one or more of biomarkers that correlate with TID.

As T1D can be an auto-immune disorder, autoimmune markers can be used to identify patients having T1D. Autoimmune markers for T1D include islet cell autoantibodies and autoantibodies to GAD (GAD65), insulin, the tyrosine phosphatases IA-2 and IA-2β, and ZnT8. Type 1 diabetes can be defined by the presence of one or more of these autoimmune markers. (Classification and Diagnosis of Diabetes: Standards of Medical Care in Diabetes. Diabetes Care 2019, 42 (Suppl.1):S13-S28). In one aspect, the methods disclosed herein are used with T1D patients having one of these markers, or having one or more of these markers.

The methods of the invention can be used with adult T1D patients who are age 18 or older, adolescent T1D patients who are age 12 to under age 18, or age 12 to age 17 or under, and in children, such as children who are age 2 to age 11.

In treatment of cardiovascular and renal disorders authors have made mention of many possible combination therapies. In some embodiments, the present methods do not involve certain combinations of medications. For example, in some embodiments, the inventive method comprises administering finerenone to a patient who is not coadministered one or more additional therapeutic agents selected from the group consisting of an SGLT2 inhibitor, an GLP1 receptor agonist, and a mineralocorticoid receptor antagonist other than finerenone. SGLT2 inhibitors are known and include without limitation canagliflozin, dapagliflozin, empagliflozin, canagliflozin, ertugliflozin, ipragliflozin, remogliflozin, sergliflozin, sotagliflozin and tofogliflozin. SGLT2 inhibitors, or gliflozins, act by inhibiting the sodium-glucose transport protein 2 (SGLT2) and thereby inhibit reabsorption of glucose in the kidney and thus lower blood sugar. SGLT2 inhibitors are used in the treatment of T2D. Glucagon-like peptide 1 (GLP-1) receptor agonists are known in the art and include without limitation dulaglutide (Trulicity®), exenatide extended release (Bydureon® bcise), exenatide (Byetta®), semaglutide (Ozempic®, Wegovy®), and liraglutide (Victoza®, Saxenda®), tirzepatide (Mounjaro®). Mineralocorticoid receptor antagonists other than finerenone include without limitation spironolactone, canrenone/K+-canrenoate, eplerenone, esaxerenone (Minnebro®), LY3437943, KBP-5074, AZD9977, and apararenone. In one embodiment, no SGLT2 inhibitor is administered. In one embodiment, SGLT2 inhibitors are excluded in the treatment.

In some embodiments, the methods of the present disclosure do not involve concomitant administration of finerenone and coadministration of both an ACEI and an ARB. In another embodiment, the methods of the present disclosure of treatment with finerenone do not involve concomitant treatment with strong CYP3A4 inhibitors, concomitant treatment with moderate/strong CYP3A4 inducers, or concomitant therapy with any renin inhibitor, sacubitril/valsartan combination, or potassium-sparing diuretic. Examples of moderate CYP3A4 inhibitors include amiodarone, aprepitant, bicalutamide, chloramphenicol, imatinib, mifepristone, norfloxacine, tacrolimus, verapamil, lapatinib, dasatinib and nilotinib.

In one aspect the invention refers to a method of preventing or treating chronic kidney disease in a patient with type I diabetes, comprising administering to the patient a therapeutically effective amount of finerenone or a hydrate, solvate, pharmaceutically acceptable salt thereof, or a polymorph thereof.

In one aspect the invention refers to finerenone or a hydrate, solvate, pharmaceutically acceptable salt thereof, or a polymorph thereof for use in a method to prevent or treat chronic kidney disease in a patient with type I diabetes, comprising administering to the patient a therapeutically effective amount thereof.

In a further aspect, the inventions to a method to delay the progression of chronic kidney disease in a patient with chronic kidney disease associated with type 1 diabetes comprising administering to the patient a therapeutically effective amount of finerenone or a hydrate, solvate, pharmaceutically acceptable salt thereof, or a polymorph thereof.

In a further aspect, the inventions to finerenone or a hydrate, solvate, pharmaceutically acceptable salt thereof, or a polymorph thereof for use in a method to delay the progression of chronic kidney disease in a patient with chronic kidney disease associated with type 1 diabetes comprising administering to the patient a therapeutically effective amount thereof. In one embodiment, the method attenuates kidney function decline and progression to end stage kidney disease or renal diseases.

In another aspect the invention refers to a method to reduce the risk of sustained UACR decline in a patient with chronic kidney disease associated with type 1 diabetes comprising administering to the patient a therapeutically effective amount of finerenone or a hydrate, solvate, pharmaceutically acceptable salt thereof, or a polymorph thereof.

In another aspect the invention refers to finerenone or a hydrate, solvate, pharmaceutically acceptable salt thereof, or a polymorph thereof for use in a method to reduce the risk of sustained UACR decline in a patient with chronic kidney disease associated with type 1 diabetes comprising administering to the patient a therapeutically effective amount thereof. In one embodiment, the method reduces the risk of end stage kidney disease or renal diseases in the patient.

In a further embodiment, the invention refers to a method to reduce UACR in a patient with chronic kidney disease associated with type 1 diabetes comprising administering to the patient a therapeutically effective amount of finerenone or a hydrate, solvate, pharmaceutically acceptable salt thereof, or a polymorph thereof.

In one embodiment, the patient has a reduction of 30% or more in UACR over baseline after six months of finerenone treatment; wherein baseline UACR is measured before or at the time of first administration of finerenone to the patient.

In one embodiment, prior to receiving finerenone, the patient has an eGFR of equal or below 45 mL/min/1.73 m², equal or above 45 mL/min/1.73 m², 45 to 60 mL/min/1.73 m², equal or below 60 mL/min/1.73 m²., or equal or above 60 mL/min/1.73 m². In one embodiment, prior to receiving finerenone, the patient has an eGFR at baseline of <45 mL/min/1.73 m². In one embodiment, prior to receiving finerenone, the patient has an eGFR at baseline of 45 to <60 mL/min/1.73 m². In one embodiment, prior to receiving finerenone, the patient has an eGFR at baseline of ≥60 mL/min/1.73 m².

In one embodiment, prior to receiving finerenone, the patient has a serum potassium of equal or below 4.5 mmol/L, or of equal or above 4.5 mmol/L.

In one embodiment, prior to receiving finerenone, the patient has
an eGFR at baseline of <45, 45 to <60, or ≥60 mL/min/1.73 m²; and/or
a UACR at baseline of <300 mg/g, 300 to ≤1000 mg/g, or >1000 mg/g; and/or
a baseline serum potassium of ≤4.5 or >4.5 mmol/L.

In one embodiment, the patient with type I diabetes is male, or female.

In one embodiment, the patient with type I diabetes is <65 or ≥65 years.

In one embodiment, the patient with type I a history of cardiovascular disease is absent or present.

In one embodiment, prior to receiving finerenone, the patient with type I diabetes has
an eGFR at baseline of <45, 45 to <60, or ≥60 mL/min/1.73 m²; and/or
a UACR at baseline of <300 mg/g, 300 to ≤1000 mg/g, or >1000 mg/g; and/or
a baseline serum potassium of ≤4.5 or >4.5 mmol/L; and/or
is male, or female; and/or
is <65 or ≥65 years; and/or
a history of cardiovascular disease is absent or present.

In one embodiment, prior to receiving finerenone, the patient has a UACR of 200 to 5000 mg/g, an eGFR of 25 to <90 ml/min/1.73 m², and has been administered a stable dosage of an ACEI or ARB for >4 weeks.

In one embodiment, prior to initiation of finerenone therapy, the patient has been administered a stable dosage of an ACEI or ARB.

In one embodiment, the patient is not coadministered one or more additional therapeutic agents selected from the group consisting of an SGLT2 inhibitor, an GLP1 receptor agonist, and a mineralocorticoid receptor antagonist other than finerenone.

In one embodiment, the patient receives finerenone at a daily dosage selected from the group consisting of 7.5 mg, 10 mg, 20 mg, and a range of 10 mg to 20 mg.

In one embodiment, the patient receives finerenone at a daily dosage selected from the group consisting of 7.5 mg, 10 mg, 20 mg, 40 mg, and a range of 10 mg to 40 mg.

In one embodiment, treatment and/or prevention includes slowing kidney disease progression in CKD associated with T1D.

### FIGURES

FIG. 1: Scheme showing study design and procedures with timeline and assessments, according to an aspect disclosed herein.

### EXAMPLES

1. Trial Conduct for Finerenone in Patients with T1D and CKD

### Study Objectives and Endpoints

The purpose of this study will be to assess efficacy and safety of finerenone (10 mg and 20 mg, OD) compared to placebo in participants with T1D and CKD. The primary variable will be the ratio of the UACR over 6 months to UACR at baseline. The primary endpoint will be to demonstrate that finerenone in addition to the existing standard of care is superior to placebo in reducing UACR over six months in participants with CKD and T1D.

Study details include a study duration per participant of 7 months, a treatment duration per participant of 6 months, and a visit frequency of approximately once every 1-3 months.

The primary clinical question of interest is whether a difference is shown between assigning finerenone and a placebo, including a difference in the effects of death and end stage renal diseases (ESRD). ESRD is defined as an occurrence of renal disease that results in initiation of chronic dialysis (hemodialysis or peritoneal dialysis) for at least 30 days or a renal transplant. In one aspect, this primary clinical question includes looking at the differences in effects irrespective of treatment discontinuation.

The most important intercurrent events (ICEs) to be measured are death and ESRD. These events are considered clinically relevant and will be handled using a composite strategy. Therefore, the treatment effects that are observed will be assessed on the primary outcome and the ICEs. A “worst case” approach is implemented to handle ICEs in terms of a composite strategy. For participants experiencing the ICE, their subsequent UACR values will be set to a “worst case” value. Given that there is no upper limit for post-baseline UACR measurements, the values will be drawn from the distribution of the largest UACR values observed during the trial (e.g. worst 10% of the UACR values). Multiple imputation methodology will be adapted for this to avoid underestimation of the standard error.

The population-level summary is a geometric mean ratio of treatment arm ‘ratios to baseline’ over the study period (i.e. average of geometric mean of treatment effect at Month 3 and Month 6 visits).

The study will also assess the safety of finerenone in addition to the standard of care compared to placebo. The safety endpoints include the number of participants with treatment-emergent adverse events (TEAE), with treatment-emergent serious adverse events (TESAE), and/or hyperkalemia. The study also has exploratory endpoints. The study is to demonstrate these exploratory endpoints: the proportion of participants with ≥30% reduction of UACR from baseline at Month 6; proportion of participants with ≥50% reduction of UACR from baseline at Month 6, change in UACR from baseline to Month 3; and change in UACR from baseline to Month 6. Another exploratory endpoint is the use of biomarkers to investigate further the study intervention, such as to investigate the mode-of-action-related effects and safety, as well as pathomechanisms relevant to renal and cardiovascular disease—including diabetes—and associated health problems.

### Study Design

This study is a double blind parallel-group intervention study in T1D patients with CKD (further eligibility characteristics discussed infra), with 2 treatment groups, including a placebo treatment group. Participants who fulfill all eligibility criteria will be randomized in a 1:1 ratio to receive finerenone or placebo, in addition to standard of care. Approximately 440 participants will be screened to achieve 220 participants randomly assigned to study intervention [110 evaluable participants per intervention group] to achieve 90% power to detect a treatment difference of at least 30%.

All participants will receive blinded study intervention of finerenone 10 mg, finerenone 20 mg, or placebo once daily on top of either an ACEI or an ARB.

The starting dose will depend on the participant's eGFR level at the Screening Visit: a lower dose of 10 mg once daily if eGFR is ≤25 to <60 mL/min/1.73 m² (CKD-EPI), or the higher (target) dose of 20 mg once daily if eGFR is ≥60 mL/min/1.73 m² (CKD-EPI). Participants who start on the lower dose of 10 mg will be up-titrated to the target dose of 20 mg from Month 1 onwards provided potassium is ≤4.8 mmol/L, and eGFR decrease is less than 30% of the last measured value. Up- and down-titration of study intervention will be based on local potassium and eGFR values. Down-titration or interruption of study intervention is permitted at any time during the study for safety reasons and study intervention will be interrupted if potassium is >5.5 mmol/L.

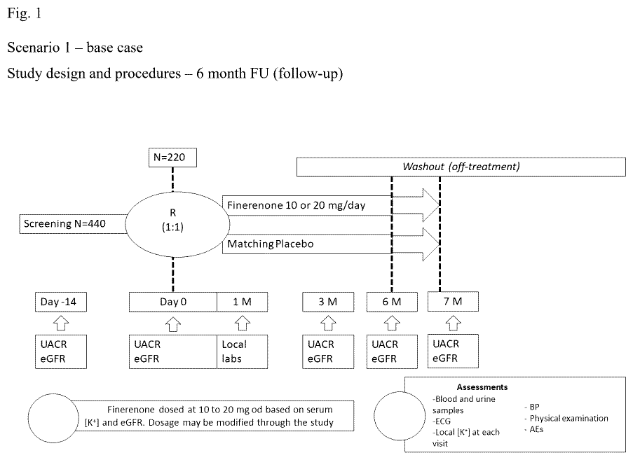

Visits will take place at Screening, Baseline, Month 1, Month 3 and Month 6 (the planned end of treatment) and at Month 7. Further details are shown in Table 1, Schedule of Activities, and FIG. 1, scheme showing study timeline.

TABLE 1
Study assessments and procedures by study period and visit are presented

| | | | | | | 4-week safety | | |
|---|---|---|---|---|---|---|---|---|
| | Screening | | | | | visit | | Follow |
| | (up to 14 | Intervention period | | | | 28 days post | Early | Up (30 |
| | days before | Day | Month | Month | Month | up-titration or | discontinuation | days after |
| Procedure | Day 0) | 0 | 1 | 3 | 6 | reinitiation | visit | last dose) |
| Visit window (days) | | | ±7 | ±7 | ±7 | ±7 | | ±7 |
| Visit number | V1 | V2 | V3 | V4 | V5 | Unscheduled | | V6 |
| Informed consent | X | | | | | | | |
| Inclusion and | X | | | | | | | |
| exclusion criteria | | | | | | | | |
| Demographics | X | | | | | | | |
| Full physical examination | | X | | | | | | X |
| Vital signs including | X | X | X | X | X | X | X | X |
| height and weight^{a} | | | | | | | | |
| Medical history | X | | | | | | | |
| Serum/Urine pregnancy | X | | | | | | | |
| test^{f} | | | | | | | | |
| (Central) UACR^{b, c} | X | X | | X | X | | X | X |
| Dispense urine pots for | X | | X | X | X | | X | |
| UACR assessments | | | | | | | | |
| (Local) Serum K+ | X | X | X | X | | X | X | |
| (Local) eGFR (serum | X | X | X | X | | X | X | |
| creatinine) | | | | | | | | |
| (Central) eGFR, K+, NA | X | X | | | X | | X | X |
| HbA1c^{c, e} | | | | | | | | |
| Biomarker (Blood/urine) | | X | | | X | | | |
| 12-lead ECG | | X | | | | | | |
| Randomization | | X | | | | | | |
| Study intervention | | X^{j} | X | X | X | | X | |
| dispensation/ | | | | | | | | |
| accountability^{d} | | | | | | | | |
| AE/SAE/OE/Con Meds | | β========================================à | | | | | | X |
| review | | | | | | | | |
| Visit window (days) | | | ±7 | ±7 | ±7 | ±7 | | ±7 |

One month is counted as 30 days
ECG: Electrocardiogram;
eGFR: Estimated glomerular filtration rate;
ICF: Informed consent form;
K+: Potassium;
UACR: Urine albumin-to-creatinine ratio;
V: Visit EQ5D: EuroQoL 5-dimension 5-level questionnaire,
HbA1c: glycated haemoglobin,
WOCBP = Women of child-bearing potential
^{a}Height will be measured at Screening only.
^{b}At Visit 1, first morning void urine samples to be collected on three consecutive days at the participant's home. At the other visits, two consecutive samples can be collected ±7 days from the visit date.
^{c}One re-assessment (central laboratory) of eGFR, K+ and/or UACR is allowed at the Screening visit.
^{d}First study intervention at study site.
Check footnote for dual off treatment eGFRs
^{f}WOCBP only. Urine or serum, as required by local regulations
The planned participant treatment duration will be 6 months and the planned follow-up time 7 months.
Additional visits will take place 4 weeks after each up-titration and after restart of study intervention following treatment interruption for >7 days.

UACR as the primary endpoint measure and serum potassium as the key safety parameter as described will be assessed according to the visit schedule. UACR will be collected at screening, baseline, Month 3 at the end of treatment Month 6. eGFR will be collected at all specified UACR collection points, and additionally at Month 1 to assess up-titration and at Month 7, to assess kidney function 4 weeks after treatment discontinuation.

UACR is a measurement of albuminuria, a predictor of long-term renal and cardiovascular adverse outcomes in patients with type 2 diabetes. (Fox et al. Lancet. 2012 Nov. 10; 380(9854):1662-73, Heerspink et al. Lancet Diabetes Endocrinol. 2019; 7(2):128-39). Post-hoc analyses of randomized clinical trials suggest that a meaningful reduction in albuminuria may translate to protection from declining renal function and cardiovascular events in patients with diabetic kidney disease. (Heerspink et al. Eur J Prev Cardiol. 2014 March; 21(3):299-309, Levey et al. Am J Kidney Dis. 2020 January; 75(1):84-104). For every 30% reduction in albuminuria, one can expect the risk of ESRD to be decreased by 23.7% (95% confidence interval 11.4%-34.2%; p=0.001). (Heerspink and Gansevoort Clin J Am Soc Nephrol. 2015 Jun. 5; 10(6):1079-88). Changes in UACR can be detected early in response to treatment.

### Eligibility

Inclusion Criteria for Participants:
1. Age: Participant is ≥18 years of age.
2. Participant has a diagnosis of T1D (ADA 2020 definition), i.e., T1D continuously treated with insulin, started within one year from diagnosis. If the onset was after age 35, the participant has the presence of one or more of the following:
   Circulating T1D-associated autoantibodies
   History of hospitalization for diabetic ketoacidosis
   Plasma C-peptide below the limit of detection with standard assay (with current blood glucose >100 mg/dl).
3. Participant has HbA1c at Screening Visit <10% (central assessment)
4. K⁺≤4.8 mmol/L at Screening Visit (local assessment)
5. Participant has a clinical diagnosis of CKD and fulfills both the criteria (central assessment):
   eGFR≥25 and <90 mL/min/1.73 m² at Screening visit
   UACR≥200 mg/g (22.6 mg/mmol)—to <5000 mg/g (565 mg/mmol) at the Screening visit,
6. Participant treated with a stable (preferably without any change in the dosage for at least 4 weeks prior to Screening visit) dose of ACEI or ARB (but not both) at Screening visit. Preferably the stable dose of ACEI or ARB is unchanged throughout the course of the study. Table 2 discusses the patient population in detail.

TABLE 2
Patient population

| Key inclusion criteria | Key exclusion criteria |
|---|---|
| Aged ≥18 years | T2D |
| T1D* | Serum [K+] >4.8 mmol/l at |
| (with HbA1c <10% at | screening visit |
| screening visit**) | |
| eGFR 25-90 ml/min/1.73 | BP >160/100 mmHg or SBP <90 |
| m²*** and UACR ≥200-5000 | mmHg at baseline visit |
| mg/g⁺ at screening visit | |
| Treated with stable dose of | Treatment with the following |
| ACEi or ARB for >1 month | medications⁺⁺: |
| prior to screening visit | SGLT-2i, SGLT-1/2i or GLP1-RA |
| | Finerenone or another MRA |
| | Chronic HFrEF and persistent symptoms |
| | (NYHA class II-IV) at run-in visit |
| | Recent CV or PAD event within 3 |
| | months⁺⁺ or invasive procedure |
| | within 4 weeks (PCI, CABG, limb |
| | intervention) ⁺⁺⁺ |

*T1D diagnosed according to ADA guidelines; American Diabetes Association. Diabetes Care 2022
**Historical values of HbA1C within 3 months of screening visit accepted
***eGFR calculated according to CKD-EPI 2021 formula based on creatinine.
⁺Mean value from three morning void samples
BP, blood pressure; CKD-EPI, chronic kidney disease epidemiology collaboration; MRA, mineralocorticoid receptor antagonist; SBP, systolic blood pressure; SGLT-1/2i, sodium-glucose co-transporter-1 and -2 inhibitor; T1/2D, type 1/2 diabetes; GLP1-RA glucagon-like peptide 1 receptor antagonist, ACEi angiotensin-converting enzyme inhibitors, ARB angiotensin II receptor blocker, NYHA New York Heart Association, CV cardiovascular, PAD peripheral artery disease, PCI percutaneous intervention, CABG coronary artery bypass graft
⁺⁺CV event within 3 months prior to screening (heart failure decompensation, myocardial infarction, stroke, transient ischemic attack, pulmonary embolism, elective coronary artery bypass grafting) or percutaneous coronary intervention within 4 weeks prior to screening
⁺⁺⁺ Open or percutaneous intervention concerning Peripheral Artery Disease within 4 weeks prior to screening

Participants are excluded from the study if any of the following medical conditions apply: T2D, other known cause of CKD than TID, having a kidney transplant, symptomatic heart failure with reduced ejection fraction with class 1A indication for MRAs, Addison's disease, hospitalization due to a CV event within 4 weeks prior to Screening visit (heart failure decompensation, acute coronary syndrome, stroke, transient ischemic attack, acute limb ischemia), acute kidney injury requiring dialysis within 24 weeks prior to Screening visit, active cancer, liver disease (Child-Pugh C, determined using the Child Pugh score grading the severity of liver disease adapted from Pugh et al. Br J Surg. 1973 August; 60(8):646-9), and mean blood pressure (BP)>160/100 mmHg or mean systolic BP (SBP)<90 mmHg at Screening visit. Participants are also excluded from the study if they have any of the following prior/concomitant therapies: (1) concomitant dual therapy with both an ACEI and an ARB that is not discontinued at least 8 weeks prior to the Screening visit, (2) current or previous (within 8 weeks prior to the Screening visit) treatment with a SGLT2/1 inhibitor, a GLP-1 receptor agonist, or MRAs other than finerenone (such as eplerenone, spironolactone, canrenone, esaxerenone, AZD9977 and KBP-5074 (ocedurenone)), (3) concomitant treatment with strong CYP3A4 inhibitors not stopped by seven days prior to randomization; (4) concomitant treatment with moderate/strong CYP3A4 inducers not stopped by seven days prior to randomization, and (5) concomitant treatment with any renin inhibitor, sacubitril/valsartan combination, or potassium-sparing diuretic that is not discontinued at least 8 weeks prior to the Screening visit.

### Statistical Methods and Sample Size

UACR will be analyzed assuming a lognormal distribution. Assumptions for the UACR ratios to baseline at 6 months are based on the FIDELITY UACR mixed model restricted to patients with UACR>=200 mg/g and eGFR between 25 and 90 ml/min/1.73 m².

It will be assumed that the effect of placebo on UACR is non-existent so for sample size calculation we will assume a UACR ratio to baseline after 6 months of 1.00 for the placebo group.

In the FIDELITY restricted population, the UACR ratio to baseline after 4 months was 0.61 and 0.53 after 12 months in the finerenone group. The value, using interpolation, at 6 months was estimated to be 0.59. As a conservative approach, we will assume the treatment ratio (finerenone/placebo) over 6 months to be 0.7 (i.e., the difference in UACR ratio to baseline between finerenone and placebo to be 30%). The standard deviation was calculated to range from 0.75 and 1.0 between month 4 and 12. Using linear interpolation, at month 6 it was assumed to be approximately 0.8.

A sample size of 214 [107 per group] achieves 90% power at a two-sided significance level of 5%, to reject the null hypothesis of treatment ratio equal to 1 assuming the true treatment ratio of 0.7 and the SD to be 0.8. To account for the potential loss of information in applying the “worst case” approach for the primary estimand, the sample size will be boosted by 2.6% (the calculated cumulative incidence of death, kidney failure from the pool of FIDELIO-DKD and FIGARO-DKD) to ensure the desired power is achieved. Therefore, the sample size is increased to 220 participants [110 per group].

### Safety

The methods and products of the invention concern dosages that are clinically proven safe and effective in T2D. Adverse events that impact whether the inventive therapy is safe are hyperkalemia, symptomatic hypotension, hyponatraemia, acute kidney injury, and severe hypoglycaemia. Intercurrent events include death and kidney failure defined as eGFR<15 ml/min/1.73 m² or renal replacement therapy (peritoneal-hemodialysis or kidney transplantation). Important safety endpoints are shown in Table 3.

TABLE 3
Safety endpoints

The decline of eGFR is measured by the slope of eGFR from baseline
to month 7 which is 4 weeks after the last on-treatment study visit (off-
treatment effect) TBC
eGFR decline >30% from baseline to 30 days
Incidences (n, %) of:
Hyperkalaemia, symptomatic hypotension, hyponatraemia, acute kidney
injury, severe hypoglycaemia
Monitoring of AEs, ECG at baseline and in case of K+ >6.0 mmol/L,
laboratory and vital signs

### Other Analysis

### Further Subgroup Analyses

Exploratory subgroup analysis will be performed for the primary efficacy variables. The list of key subgroups and other subgroups analyzed is specified below. Analyses will include descriptive statistics, estimated ratios to baseline with 95%-CIs.

Subgroups can be:
eGFR category at baseline (eGFR<45, 45 to <60, ≥60 mL/min/1.73 m²).
UACR category at baseline (<300 mg/g, 300 to ≤1000 mg/g, ≥1000 mg/g).
Baseline serum potassium (≤4.5, >4.5 mmol/L)·
Race (white, black, Asian, other)
Sex (male, female)
Age group (<65, ≥65 years).
History of CV disease (absent, present)

### 2. Measurements of Finerenone Polymorph

### Lattice Constants of Compound of the Formula (I) in Crystalline Form of Polymorph I

Polymorph I
Crystal system orthorhombic
Space group P2(1) 2(1) 2(1)
Molecules per unit
cell 4
Length of axis a [Å] 7.8610(3)
Length of axis b [Å] 11.7797(6)
Length of axis c [Å] 20.1792(8)
α [°] 90
β [°] 90
γ [°] 90
Calculated density at
100 K [g cm-3] 1.345
   Measuring Parameters of the x-Ray Diffractometry for the Measurement of Compound of the Formula (I) in Crystalline Form of Polymorph I

| | |
|---|---|
| Data set name | 2429-08a r2 |
| Scan axis | 2Theta-Omega |
| Start position [°2Th.] | 2.0000 |
| End position [°2Th.] | 37.9900 |
| Type of divergence screen | Fixed |
| Size of divergence screen [°] | 1.0000 |
| Measurement temperature [° C.] | 25 |
| Anode material | Cu |
| K-Alpha1 [Å] | 1.54060 |
| Generator setting | 35 mA, 45 kV |
| Diffractometer type | Transmission diffractometer |
| Goniometer radius [mm] | 240.00 |
| Focus-div. screen gap [mm] | 91.00 |
| Primary beam monochromator | Yes |
| Sample rotation | Yes |

Peak maximum
[2 Theta]
Polymorph I
8.5
11.4
11.9
13.4
14.1
14.8
15.0
15.4
16.0
17.2
18.5
19.0
19.8
20.5
20.8
22.1
22.7
23.0
23.1
23.6
23.9
24.6
24.9
25.2
25.6
26.0
26.5
27.1
27.3
28.3
28.5
28.8
29.6
30.1
30.6
31.5
31.9
32.4
32.9
33.1
33.4
33.7
34.5
34.7
35.0
35.8
36.2
36.5
37.2
37.4

### Measuring Conditions for the IR and Raman Spectroscopy for the Measurement of the Compound of the Formula (I) in Crystalline Form of Polymorph I:

| IR: | | |
|---|---|---|
| Instrument | Perkin Elmer Spectrum One | |
| Number of scans | 32 | |
| Resolution | 4 | cm⁻¹ |
| Technique | Diamond ATR unit | |
| Raman: | | |
| Instrument | Bruker Raman RFS 100/S | |
| Number of scans | 64 | |
| Resolution | 2-4 | cm⁻¹ |
| Laser Power | 350 | mW |
| Laser wavelength | 1064 | nm |

Band maximum [cm⁻¹]

| 1R-ATR | Raman |
|---|---|
| Polymorph I | Polymorph I |
| 3475 | 3074 |
| 3416 | 2997 |
| 3366 | 2970 |
| 3074 | 2941 |
| 2992 | 2920 |
| 2952 | 2836 |
| 2835 | 2231 |
| 2230 | 1659 |
| 1681 | 1641 |
| 1658 | 1623 |
| 1606 | 1601 |
| 1572 | 1577 |
| 1485 | 1487 |
| 1464 | 1443 |
| 1454 | 1383 |
| 1431 | 1362 |
| 1420 | 1327 |
| 1407 | 1303 |
| 1381 | 1267 |
| 1355 | 1230 |
| 1341 | 1191 |
| 1325 | 1161 |
| 1303 | 1123 |
| 1285 | 1093 |
| 1267 | 1032 |
| 1255 | 991 |
| 1229 | 883 |
| 1222 | 827 |
| 1161 | 810 |
| 1136 | 759 |
| 1097 | 734 |
| 1031 | 708 |
| 991 | 671 |
| 976 | 613 |
| 967 | 528 |
| 924 | 505 |
| 909 | 471 |
| 875 | 442 |
| 847 | 346 |
| 827 | 320 |
| 810 | 297 |
| 776 | 186 |
| 758 | 155 |
| 746 | 114 |
| 733 | |
| 723 | |
| 706 | |
| 697 | |
| 670 | |

1. A method of preventing or treating chronic kidney disease in a patient with type I diabetes, comprising administering to the patient a therapeutically effective amount of finerenone or a hydrate, solvate, or pharmaceutically acceptable salt thereof, or a polymorph thereof.

2. The method of claim 1, wherein prior to receiving finerenone, the patient with type I diabetes has
(1) albuminuria,
(2) a UACR of equal to or above 150, 200, 250, 300, 350, 400, 450 or 500 mg/g, and/or
(3) a UACR of 30 to 5000, 200 to 5000, 200 to 4000, 200 to 3000, or 200 to 2000 mg/g.

3. The method of claim 1, wherein prior to receiving finerenone, the patient with type I diabetes has a UACR of
(1) equal to or below 200 mg/g, or
(2) equal to or above 200 mg/g, or
(3) equal to or below 300 mg/g, or
(4) equal to or above 300 mg/g, or
(5) 300 to 1000 mg/g, or
(6) equal to or below 1000 mg/g, or
(7) equal to or above 1000 mg/g, or
(8) 200 to 5000 mg/g, or
(9) 200 to 1000 mg/g, or
(10) equal to or below 5000 mg/g.

4. The method of claim 1, wherein prior to receiving finerenone, the patient with type I diabetes has
(1) an eGFR of equal to or above 5, 10, 15, 20, 25, 30, 35, 40, 45, 50, 55, 60, 65, 70, or 75 ml/min/1.73 m², or
(2) an eGFR of from 15-90, 20-90, 25-90,30-90, 60-90, 25-75, or 25-60 mL/min/1.73 m².

5. The method of claim 1, wherein prior to receiving finerenone, the patient with type I diabetes has an eGFR of
(1) equal to or below 25 mL/min/1.73 m², or
(2) equal to or above 25 mL/min/1.73 m², or
(3) equal to or below 45 mL/min/1.73 m², or
(4) equal to or above 45 mL/min/1.73 m², or
(5) 45 to 60 mL/min/1.73 m², or
(6) 45 to 90 mL/min/1.73 m², or
(7) 25 to 60 mL/min/1.73 m², or
(8) 25 to 90 mL/min/1.73 m², or
(9) equal or below 60 mL/min/1.73 m², or
(10) equal or above 60 mL/min/1.73 m², or
(11) equal or below 90 mL/min/1.73 m².

6. The method of claim 1, wherein prior to receiving finerenone, the patient with type I diabetes has a UACR of 200 to 5000 mg/g, an eGFR of 25 to <90 ml/min/1.73 m², and has been administered a stable dosage of an ACEI or ARB for >4 weeks.

7. The method of claim 1, wherein prior to initiation of finerenone therapy, the patient has been administered a stable dosage of an ACEI or ARB.

8. The method of claim 1, wherein the patient is not coadministered one or more additional therapeutic agents selected from the group consisting of an SGLT2 inhibitor, an GLP1 receptor agonist, and a mineralocorticoid receptor antagonist other than finerenone.

9. The method of claim 1, wherein the patient receives finerenone at a daily dosage selected from the group consisting of 7.5 mg, 10 mg, 20 mg, 40 mg, a range of 10 mg to 20 mg, and a range of 10 mg to 40 mg.

10. The method of claim 1, wherein the patient receives finerenone in a daily amount of 0.25 mg to 80 mg.

11. The method of claim 2, wherein the method delays the progression of chronic kidney disease in the patient with chronic kidney disease associated with type 1 diabetes.

12. The method of claim 2, wherein the method reduces the risk of sustained UACR decline in the patient with chronic kidney disease associated with type 1 diabetes.

13. The method of claim 1, wherein finerenone is a compound of the formula (I) in crystalline form of polymorph I 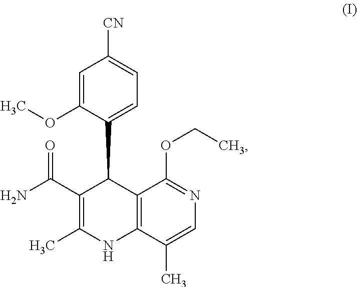 [SMILES: CCOc1ncc(C)c2c1[C@H](c1ccc(C#N)cc1OC)C(C(N)=O)=C(C)N2]
wherein the x-ray diffractogram of the compound exhibits peak maxima of the 2 theta angle at 8.5, 14.1, and 19.0.

14. The method of claim 1, wherein finerenone is the compound of formula (I) in crystalline form of polymorph I, wherein the x-ray diffractogram of the compound further exhibits any of peak maxima of the 2 theta angle at 17.2, 20.5, 25.6, and 26.5.

15. The method of claim 1, wherein finerenone is the compound of the formula (I) in crystalline form of polymorph I according to claim 14, wherein the IR spectrum of the compound exhibits any one of band maxima at 3475, 2230, 1681, 1658, 1606, 1572, 1485, 1255, 1136 and 1031 cm⁻¹.

16. The method of claim 2, wherein finerenone is a compound of the formula (I) in crystalline form of polymorph I 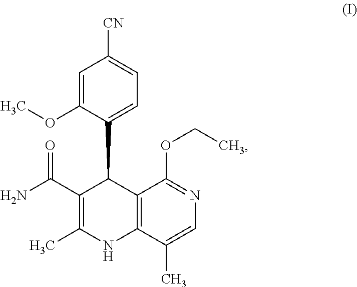 [SMILES: CCOc1ncc(C)c2c1[C@H](c1ccc(C#N)cc1OC)C(C(N)=O)=C(C)N2]
wherein the x-ray diffractogram of the compound exhibits peak maxima of the 2 theta angle at 8.5, 14.1, and 19.0.

17. The method of claim 6, wherein finerenone is a compound of the formula (I) in crystalline form of polymorph I 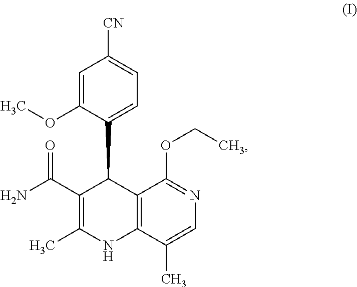 [SMILES: CCOc1ncc(C)c2c1[C@H](c1ccc(C#N)cc1OC)C(C(N)=O)=C(C)N2]
wherein the x-ray diffractogram of the compound exhibits peak maxima of the 2 theta angle at 8.5, 14.1, and 19.0.

18. The method of claim 2, wherein prior to initiation of finerenone therapy, the patient has been administered a stable dosage of an ACEI or ARB.

19. The method of claim 6, wherein prior to initiation of finerenone therapy, the patient has been administered a stable dosage of an ACEI or ARB.

20. The method of claim 2, wherein the patient is not coadministered one or more additional therapeutic agents selected from the group consisting of an SGLT2 inhibitor, an GLP1 receptor agonist, and a mineralocorticoid receptor antagonist other than finerenone.
